(19) United States
(12) Patent Application Publication
(10) Pub. No.: US 2004/0176496 A1
(43) Pub. Date: Sep. 9, 2004

(54) Light-curing dental adhesive compositions

(76) Inventors: Dong Keun Han, Seoul (KR); Kyu Hyun Baek, Goyang-si (KR); Sang Soon Park, Seoul (KR); Kwang Soo Park, Seoul (KR)

Correspondence Address: JONES DAY, 222 EAST 41ST ST, NEW YORK, NY, 10017, US

(21) Appl. No.: 10/699,537

(22) Filed: Oct. 31, 2003

(30) Foreign Application Priority Data
Mar. 4, 2003 (KR) 10-2003-0013271

(51) Int. Cl. A61K 6/00

(52) U.S. Cl. 522/183; 523/120

(57) ABSTRACT

Disclosed is light-curing dental adhesive compositions i) based on the multifunctional prepolymer mixture of 2,2-bis-(4-(2-hydroxy-3-methacryloyloxypropoxy)phenyl)propane (“Bis-GMA”) and multifunctional prepolymer formed by substituting hydrogen atoms in hydroxyl group with methacrylate groups in the Bis-GMA molecules; and ii) comprising an adhesive monomer having both a hydrophobicity and hydrophilicity, a hydrophilic monomer, a photoinitiation system and a diluent.

### FIELD OF THE INVENTION

The present invention relates to light-curing dental adhesive compositions that are applied to the mouth and have enhanced mechanical and physical properties to keep the properties for long time sustainability after operation.

More specifically, the present invention relates to new light-curing dental adhesive compositions i) based on the multifunctional prepolymer mixture of 2,2-bis-(4-(2-hydroxy-3-methacryloyloxypropoxy)phenyl)propane (“Bis-GMA”) which is used as a base prepolymer of conventional dental adhesive compositions and multifunctional prepolymer formed by substituting hydrogen atoms in hydroxyl group with methacrylate groups in the Bis-GMA molecules; and ii) comprising an adhesive monomer having both a hydrophobicity and hydrophilicity, a hydrophilic monomer, a photoinitiation system and a diluent.

### BACKGROUND OF THE INVENTION

The key factors that determine the performances of a dental adhesive are constituents of the adhesive, a photoinitiation system and a diluent. Generally, dental adhesion is divided into enamel adhesion and dentine adhesion.

In enamel adhesion, a soft surface of enamel is changed to a rough surface by acid corrosion to facilitate capillary phenomenon development therein thereby making permeation of a dental restoration material into minute pores, by which enhanced sustainability can be obtained. Bis-GMA has been developed and used as an enamel adhesive component and the superior strength and advantages are described in U.S. Pat. Nos. 4,102,856 and 4,131,729, etc. However, since Bis-GMA is hydrophobic and has a high viscosity, there are problems that it is required to use hydroxyethyl methacrylate (HEMA) having a high hydrophilicity as well as triethylene glycol dimethacrylate (TEGDMA) for lowering the viscosity.

Furthermore, in case of dentine adhesion, enamel is non-live tissue while dentine is considered as being extension of the dental pulp anatomically and histologically. Therefore, any materials, which are applied to dentine, should have no biological detrimental effects and no threat to biological stability of teeth. Enamel comprises 96% of calcium salt on the basis of weight and 4% of water on the basis of volume while dentine comprises 70% of inorganic apatite on the basis of weight and 22% of water on the basis of volume. Such structural differences give rise to great influence on the dentine adhesion, which makes the dentine adhesion difficult. Since it has been confirmed that the adhesion strength could be enhanced by using a highly hydrophilic primer and an adhesive monomer having both the hydrophilicity and the hydrophobicity, various compounds have been proposed.

JP-B 53-33687 and JP-B 54-10986 disclose compositions comprising carbonyl compounds such as an α-diketone and an amine although the compositions show the insufficient waterproof property and adhesion strength. JP-A 56-120610 proposes a light-curing composition comprising a vinyl monomer having an acidic group, an α-diketone and an aromatic sulfinate, which composition shows the low adhesive durability. JP-B 61-3684 discloses a composition comprising a monomer including a carboxylic anhydride, an organic peroxide, an amine and an aromatic sulfinate although the composition shows the insufficient adhesion strength. Thereafter, compositions comprising a carboxylic acid containing monomer and a phosphate based adhesive monomer have been proposed to have the improved color tone stability and adhesive strength.

A photoinitiation system is important for showing a sufficient function of a dental adhesive. A light-curing composition is exposed to visible rays that are unharmful to the human body to form a radical from a photoinitiator and a reductant. Said radical initiates polymerization of a monomer for curing the composition. Polymerization primarily occurs by exposure of a photoinitiator such as α-diketone aliphatic and aromatic carbonyl compound and tert-amine reductant (catalyst) to the visible ray under a wavelength ranging from 400 to 500 nm.

Furthermore, a diluent is important to facilitate a diffusion of the dental adhesive composition into a dental tissue. The diluent should function to solubilize the composition, mix the composition with a water in teeth, remove gradually the water from teeth surface as it evaporates and make the composition absorbed into teeth tissue. JP-B 6-62688 proposes a composition comprising an aqueous volatile organic solvent such as ethanol, however it does not show a sufficient adhesive performance.

### SUMMARY OF THE INVENTION

It is an object of the invention to provide light-curing dental adhesive compositions i) comprising a multifunctional prepolymer, a diluent, a photoinitiation system (a photoinitiator and a reductant), a hydrophilic monomer and an adhesive monomer, and ii) showing improved physical and mechanical properties, and adhesive performance in comparison with conventional dental adhesive compositions based on Bis-GMA.

Bis-GMA has been most frequently used as a prepolymer for dental adhesive because of its superior physical properties, such as its high strength after curing. The Bis-GMA molecule has two hydroxyl groups and two methacrylates that can be subject to light-curing, whereas said hydroxyl groups have a property to absorb moisture, which makes the physical properties of a photocured substance reduced. Thus, if a polymerized resin is swelled by water-absorption, the binding force between it and the filler is weakened such that the filler particle is likely to separate from the resin.

Therefore, the photocured substance is weakened with respect to its physical properties, such as its strength and abrasion resistance and it may induce cytotoxicity.

The present inventors have conducted extensive research to improve the aforesaid problems of conventional photo-cured dental adhesive material prepared solely from Bis-GMA prepolymers. We have found that if a prepolymer mixture of Bis-GMA and trifunctional methacrylate prepolymer (Tri-GMA) and/or tetrafunctional methacrylate prepolymer (Tetra-GMA) that is formed by substituting at least one hydrogen atom in the two hydroxyl groups with methacrylate groups in the Bis-GMA molecule, is used together with a conventional adhesive monomer in preparing a light-curing dental adhesive material, improved physical and mechanical properties, and low polymerization shrinkage of photo-cured substance can be obtained.

Therefore, the invention relates to light-curing dental adhesive compositions i) comprising a multifunctional prepolymer mixture of Tri-GMA and/or Tetra-GMA with Bis-GMA, an adhesive monomer, a hydrophilic monomer, a photoinitiation system and a diluent, and ii) showing improved physical and mechanical properties, and adhesive performance.

More specifically, the invention provides light-curing dental adhesive compositions, which show improved physical and mechanical properties, and adhesive performance, comprising:

(a) 1 to 50 wt % of the prepolymer mixture selected from a group consisting of a mixture of 2,2-bis-(4-(2-hydroxy-3-methacryloyloxypropoxy)phenyl)propane (Bis-GMA) of formula 1 with trifunctional methacrylate (Tri-GMA) of formula 2, a mixture of Bis-GMA with tetrafunctional methacrylate (Tetra-GMA) of formula 3 and a mixture of Bis-GMA, Tri-GMA and Tetra-GMA,

(b) 1 to 40 wt % of an adhesive monomer;

(c) 1 to 10 wt % of a hydrophilic monomer;

(d) 0.1 to 10 wt % of a photoinitiation system; and

(e) 10 to 60 wt % of a diluent,

wherein the wt % of all the components are based on the total weight of the composition;

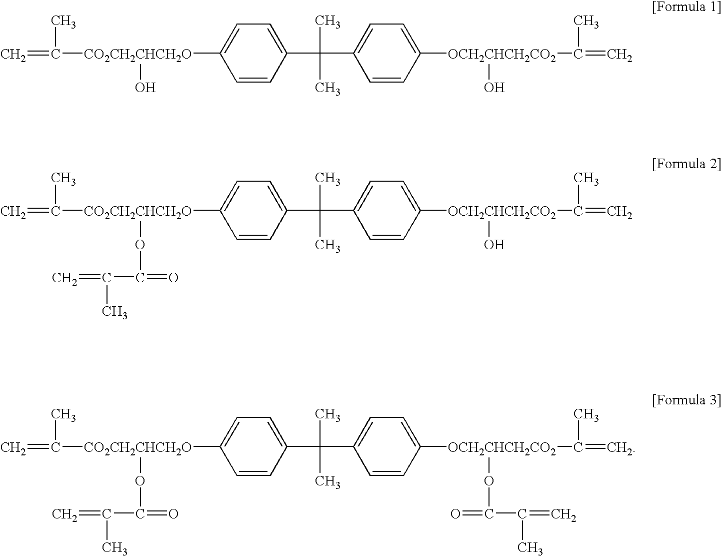

Other objects and advantages of the invention will be clarified in the following detailed description of the invention provided below.

### DETAILED DESCRIPTION OF THE PRESENT INVENTION

The present invention will now be described in detail.

According to the first embodiment, there are provided light-curing dental adhesive compositions comprising 1 to 50 wt % of the prepolymer mixture of Bis-GMA of formula 1 with Tri-GMA of formula 2, 1 to 40 wt % of an adhesive monomer, 1 to 10 wt % of a hydrophilic monomer, 0.1 to 10 wt % of a photoinitiation system and 10 to 60 wt % of a diluent, wherein the wt % of all the components are based on the total weight of the composition. The weight ratio of Bis-GMA of formula 1 to Tri-GMA of formula 2 is 95:5 to 5:95.

According to the second embodiment, there are provided light-curing dental adhesive compositions comprising 1 to 50 wt % of the prepolymer mixture of Bis-GMA of formula 1 with Tetra-GMA of formula 3, 1 to 40 wt % of an adhesive monomer, 1 to 10 wt % of a hydrophilic monomer, 0.1 to 10 wt % of a photoinitiation system and 10 to 60 wt % of a diluent, wherein the wt % of all the components are based on the total weight of the composition. The weight ratio of Bis-GMA of formula 1 to Tetra-GMA of formula 3 is 95:5 to 5:95.

According to the third embodiment, there are provided light-curing dental adhesive compositions comprising 1 to 50 wt % of the prepolymer mixture of Bis-GMA of formula 1 with Tri-GMA of formula 2 and Tetra-GMA of formula 3, 1 to 40 wt % of an adhesive monomer, 1 to 10 wt % of a hydrophilic monomer, 0.1 to 10 wt % of a photoinitiation system and 10 to 60 wt % of a diluent, wherein the wt % of all the components are based on the total weight of the composition. The prepolymer mixture consists of 90 to 5 wt % of Bis-GMA of formula 1, 90 to 5 wt % of Tri-GMA of formula 2, and 90 to 5 wt % of Tetra-GMA of formula 3 on the basis of the total weight of the prepolymer mixture.

In accordance with the light-curing dental adhesive compositions of the invention, Tri-GMA of formula 2 and Tetra-GMA of formula 3, constituting the prepolymer mixture, may be synthesized by substituting at least one hydrogen atoms in the two hydroxyl groups with methacrylate group in Bis-GMA molecule of formula 1. Thus, scheme 1 shows that Tri-GMA and Tetra-GMA may be quantitatively synthesized by reacting Bis-GMA with methacryloyl chloride in the presence of an organic amine, for example, triethylamine.

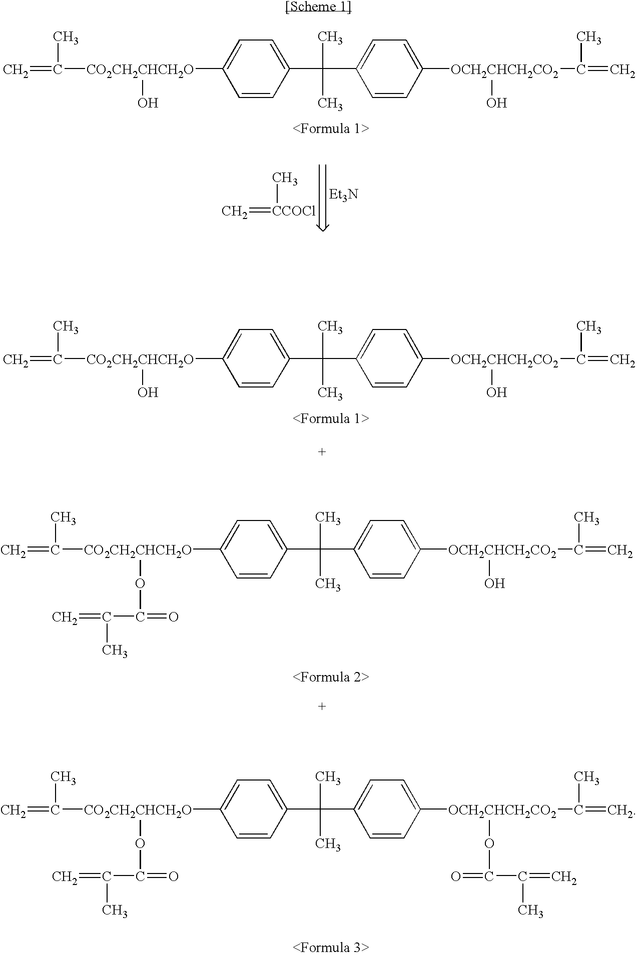

The synthesized multifunctional prepolymer mixture is separated into Bis-GMA, Tri-GMA and Tetra-GMA, through a column using a developer of mixture of ethyl acetate and n-hexane (50:50 weight ratio).

In accordance with the present invention, the light-curing dental adhesive compositions comprise a prepolymer mixture in an amount of 1 to 50 wt % of the total weight of the composition. If the prepolymer mixture of less than 1 wt % is present, the effect by itself is small and if the mixture of more than 50 wt % is present, the viscosity of the composition can be too high.

The compositions according to the present invention comprise the adhesive monomers containing functional groups such as carboxylic acid and its derivative, phosphoric acid group and sulfonic acid group, etc. in order to enhance the adhesion strength of the composition on both the teeth and a dental restoration material.

The monofunctional polymerizable monomers having a carboxylic acid or carboxylic anhydride group in the molecule include monocarboxylic acid, dicarboxylic acid, tricarboxylic acid, tetracarboxylic acid, polycarboxylic acid and their anhydrides. The example of the compounds which can be used in the invention includes (meth)acrylic acid, maleic acid, p-vinylbenzoic acid, 11-(meth)acryloxy-1,1-undecandicarboxylic acid (MAC-10), 1,4-di(meth)acryloyloxyethylpyromellitic acid, 6-(meth)acryloyloxyethyl naphthalene-1,2,6-tricarboxylic acid, 4-(meth)acryloyloxymethyltrimellitic acid and its anhydride, 4-(meth)acryloxyethyltrimellitic acid (4-MET) and its anhydride (4-META), 4-(meth)acryloxybuthyltrimellitic acid and its anhydride, 4-[2-hydroxy-3-(meth)acryloyloxy]buthyltrimellitic acid and its anhydride, 2,3-bis(3,4-dicarboxybenzoyloxy)propyl(meth)acrylate, 2-, 3- or 4-(meth)acryloyloxybenzoic acid, N—O-di(meth)acryloyloxytyrosine, O-(meth)acryloyloxytyrosine, N-(meth)acryloyloxytyrosine, N-(meth)acryloyloxyphenylalanine, N-(meth)acryloyl-p-aminobenzoic acid, N-(meth)acryloyl-O-aminobenzoic acid, an addition product of glycidyl(meth)acrylate with N-phenylglycine or N-tolylglycine, 4-[(2-hydroxy-3-(meth)acryloyloxypropyl)amino]phthalic acid, 3- or 4-[N-methyl-N-(2-hydroxy-3-(meth)acryloyloxy)amino]phthalic acid, (meth)acryloylamino salicylic acid and (meth)acryloyloxy salicylic acid. Among them, MAC-10 and 4-MET or its anhydride (4-META) are preferred.

The multifunctional polymerizable monomers having at least two carboxylic groups in the molecule which can be used as an adhesive monomer includes dicarboxylic acid, tricarboxylic acid, tetracarboxylic acid and their derivatives such as an addition product of 2-hydroxyethyl(meth)acrylate with pyromellitic dianhydride (PMDA), 2-hydroxyethyl(meth)acrylate 2 mole and (meth)acrylate of maleic anhydride or 3,3,4,4′-benzophenone tetracarboxylic dianhydride (BTDA) or 3,3,4,4-diphenyltetracarboxylic dianhydride, and 2-(3,4-dicarboxylbenzoyloxy) 1,3-di(meth)acryloyloxypropane.

The polymerizable monomers having at least one phosphoric acid groups in the molecule include 2-(meth)acryloyloxyethyl phosphate, 2- and 3-(meth)acryloyloxypropyl phosphate, 4-(meth)acryloyloxybuthyl phosphate, 6-(meth)acryloyloxyhexyl phosphate, 8-(meth)acryloyloxyoctyl phosphate, 10-(meth)acryloyloxydecyl phosphate, 12-(meth)acryloyloxydodecyl phosphate, bis[2-(meth)acryloyloxyethyl]phosphate, bis[2-(meth)acryloyloxyethylphenyl]phosphate, bis[2-(methyl)acryloyloxyethyl p-methoxyphenyl]phosphate, etc. Especially, 2-methacryloyloxyethyl phosphate, 10-methacryloyloxydecyl phosphate and bis-methacryloyloxyethyl phosphate are preferred.

The polymerizable monomers having sulfonic acid group in the molecule include 2-sulfoethyl(meth)acrylate, 2- or 1-sulfo-1 or 2-propyl(meth)acrylate, 1- or 3-sulfo-2-butyl(meth)acrylate, 3-bromo-2-sulfo-2-propyl(meth)acrylate, 3-methoxy-1-sulfo-2-propyl(meth)acrylate, 1,1-dimethyl-2-sulfoethyl(meth)acrylamide propanesulfonic acid and 2-methyl-2-(methyl)acrylamide propane sulfonic acid, etc. Among them, 2-methyl-2-methacrylamide propane sulfonic acid is preferred.

The adhesive monomers can be used alone or in combination, preferably in an amount of 1 to 40 wt % based on the total weight of the composition. If the monomers are present in an amount of less than 1 wt %, the effect by themselves is small and if the monomers of more than 40 wt % are present, the physical properties of the composition can be reduced.

The compositions of the present invention comprise 1 to 10 wt % of a hydrophilic monomer that makes their adhesion to the teeth possible in the presence of water because the teeth contain a significant amount of water and the ones suitable for use in the invention include hydroxyethyl methacrylate or hydroxypropyl methacrylate (HPMA). If the hydrophilic monomer of less than 1 wt % is present, the effect by itself is small and if the monomer of more than 10 wt % is present, the composition becomes high hydrophilic, which can reduce the physical properties of the compositions.

The dental adhesive compositions of the present invention are exposed to visible rays that are unharmful to the human body to form a radical from a photoinitiator and a catalyst. Said radical initiates polymerization of a monomer for curing the composition. Polymerization primarily occurs by exposure of a photoinitiator such as α-diketone aliphatic and aromatic carbonyl compound and tert-amine reductant to the visible ray under a wavelength ranging from 400 to 500 nm. The photoinitiation system consists of a photoinitiator and a reductant. The photoinitiator is preferably camphorquinone (CQ), and added in an amount of 0.05 to 5 wt % of the total weight of the composition. If CQ is photoexcited to extract hydrogen from the reductant, the reductant practically initiates radical polymerization. The reductant such as N,N-dimethylaminoethyl methacrylate (DMAEMA) or ethyl p-dimethyl aminobenzoate (EDMAB) is added in an amount of 0.05 to 5 wt % on the basis of the total weight of the composition. If the used amount of the photoinitiator and reductant is too small, the rate of polymerization is too slow to cause an inconvenience in dental treatment.

If the used amount is too many, the rate of polymerization is fast while it is difficult to form a macromolecule, which causes weakening of the physical properties of the composition. Therefore, it is required to use the appropriate amount of them.

In accordance with the present invention, the compositions comprise a diluent to reduce the viscosity of the composition and to remove water of the teeth because of its high volatility. The suitable examples of the diluent include ethyl alcohol, acetone, water, etc. The composition comprises the diluent in an amount of 10 to 60 wt % of the total weight of the composition.

The physical properties of the specimen from the resulting dental adhesive compositions are estimated as follows:

1) Photoconversion

Photopolymerization efficiency caused by the visible ray is estimated by means of infrared absorption spectroscopy. The conversion of methacrylate monomer is calculated by measuring the decreased area of the absorption band at 1638 cm⁻¹ by the aliphatic double bond on the basis of the area of the absorption band at 1690 cm⁻¹ by the aromatic ring.

2) Polymerization Shrinkage

A cylindrical specimen of the dental adhesive composition [6.0 cm (diameter)×3.3 mm (height)] is put into a transparent glass mold, and then cured using a light curing unit. A density of the specimen before and after curing is measured using a picnometer.

3) Water Absorption and Solubility

A dental adhesive composition is made into about a 6 cm (diameter)×3 mm (thickness) specimen, which is cured. The weight of the cured specimen is measured, and then the specimen is dipped into distilled water at 37° C. After every 24 or 48 hours, the specimen is then taken out, water is removed from a surface of the specimen, and the weight of the specimen is measured. Moisture absorption is calculated by the following formula.

$$Water absorption ( \% ) = \frac{ \text{(weight after dipping} - \\ \text{weight after cure before dipping)}}{\text{weight after cure before dipping}} \times 100$$

In order to measure solubility, the specimen is taken out, and water is removed from the specimen. The specimen is completely dried again in a desiccator to have uniform weight, and the weight of the specimen is measured. Solubility is calculated by the following formula.

$$Solubility ( \% ) = \frac{ \text{(weight after cure before dipping} - \\ \text{weight after dipping and complete drying)}}{\text{weight after cure before dipping}} \times 100$$

4) Radiolucency

A 13 mm [diameter]×2 mm [thickness] specimen of the composition is prepared and placed on a radioactive film together with aluminum step-panel of 2 mm thickness having a purity of 99.9%. The film is exposed to a radioactive ray (65±5 kvp, 15 mA) for 0.05 sec and developed. The radiolucency of the specimen is determined using a densitometer.

5) Adhesion Strength

A tooth specimen having a uniform thickness is cut in parallel with occlusal surface using a microtome and then a tube containing the composition is attached thereto to determine the adhesion strength using Instron.

6) Cytotoxicity

The cytotoxicity of a dental adhesive is estimated by comparing the toxicity degree according to the agar layered plate method. A specimen of 10 mm (diameter)×2 mm (thickness) is tested using polyvinylchloride [PVC, response rate: 3/4] as the positive control group and polyethylene (PE) as the negative control group. An L-929 cell suspension and an Eagle's agar medium are plated on the specimen to culture the cell in 37° C., 5% CO₂ incubator for 24 hours. The cell lysis ratio is measured in a discolored region of the specimen and is indicated in a zone index and lysis index as in Table 1, from which a response index (RI=zone index/lysis index) is calculated. Cytotoxicity is evaluated from RI value as in Table 2. The lower value means a lower toxicity.

TABLE 1
Definition of each index value

| Index | Definition |
|---|---|
| Zone Index | Discolored area |
| 0 | None permeation under the specimen |
| 1 | The limited area under the specimen |
| 2 | Area diffused from sample <0.5 cm |
| 3 | Area diffused from sample <1.0 cm |
| 4 | Area diffused from sample ≧1.0 cm, < total area |
| 5 | Area diffused from sample: total area |
| Lysis Index | Lysis area |
| 0 | None |
| 1 | <20% |
| 2 | 20-40% |
| 3 | 40-60% |
| 4 | 60-80% |
| 5 | ≧80% |

TABLE 2
Evaluation of cytotoxicity

| Scale | RI | Cytotoxicity |
|---|---|---|
| 0 | 0/0 | None |
| 1 | 1/1 | Weak |
| 2 | 2/2 to 3/3 | Medium |
| 3 | 4/4 to 5/5 | Severity |

The present invention is illustrated in detail by the examples given below. However, the examples presented here are for illustrative purposes only and should not be construed as limiting the invention.

### EXAMPLE 1

To prepare a light-curing dental adhesive composition based on a prepolymer mixture consisting of 50 wt % of Bis-GMA and 50 wt % of Tri-GMA, 20 wt % of the prepolymer mixture was mixed with 40 wt % of an adhesive monomer 4-META, 10 wt % of a hydrophilic monomer HEMA, 1.0 wt % of CQ, 1 wt % of DMAEMA and 28 wt % of acetone based on the total weight of the composition, respectively. The physical properties of the prepared adhesive composition were shown in Table 3.

### EXAMPLE 2

To prepare a light-curing dental adhesive composition based on a prepolymer mixture consisting of 75 wt % of Bis-GMA and 25 wt % of Tri-GMA, 10 wt % of the prepolymer mixture was mixed with 50 wt % of an adhesive monomer 10-methacryloyloxydecyl phosphate, 5 wt % of a hydrophilic monomer HPMA, 0.5 wt % of CQ, 0.5 wt % of EDMAB and 34 wt % of ethanol based on the total weight of the composition, respectively. The physical properties of the prepared adhesive composition were shown in Table 3.

### Example 3

To prepare a light-curing dental adhesive composition based on a prepolymer mixture consisting of 25 wt % of Bis-GMA and 75 wt % of Tri-GMA, 30 wt % of the prepolymer mixture was mixed with 25 wt % of an adhesive monomer (2-methacryloyloxyethyl phospate:bis(2-methacryloyloxyethyl) phosphate=50 wt %: 50 wt %), 15 wt % of a hydrophilic monomer HEMA, 2.0 wt % of CQ, 2 wt % of DMAEMA and 26 wt % of acetone based on the total weight of the composition, respectively. The physical properties of the prepared adhesive composition were shown in Table 3.

### EXAMPLE 4

A light-curing dental adhesive composition based on a prepolymer mixture consisting of 45 wt % of Bis-GMA, 45 wt % of Tri-GMA and 10 wt % of Tetra-GMA was prepared in the same manner as Example 1 except that 20 wt % of the prepolymer mixture and 40 wt % of MAC-10 as an adhesive monomer were added based on the total weight of the composition, respectively. The physical properties of the prepared adhesive composition were shown in Table 3.

### EXAMPLE 5

A light-curing dental adhesive composition based on a prepolymer mixture consisting of 50 wt % of Bis-GMA and 50 wt % of Tetra-GMA was prepared in the same manner as Example 3 except that 30 wt % of the prepolymer mixture and 25 wt % of 2-methyl-2-methacrylamide propane sulfonic acid as an adhesive monomer were added based on the total weight of the composition, respectively. The physical properties of the prepared adhesive composition were shown in Table 3.

### COMPARATIVE EXAMPLE 1

A light-curing dental adhesive composition based on only Bis-GMA prepolymer was prepared in the same manner as Example 1 except that 20 wt % of the Bis-GMA prepolymer was added based on the total weight of the composition. The physical properties of the prepared adhesive composition were shown in Table 3.

TABLE 3

| Physical | | | | | | Comparative |
|---|---|---|---|---|---|---|
| property | Example | | | | | example |
| factor | 1 | 2 | 3 | 4 | 5 | 1 |
| Photoconversion (%) | 51 | 50 | 48 | 46 | 47 | 45 |
| Polymerization | 2.1 | 2.5 | 2.4 | 2.4 | 2.1 | 6.5 |
| shrinkage (%) | | | | | | |
| Water absorption (%) | 15 | 14 | 14 | 13 | 13 | 21 |
| Solubility (μg/mm³) | 1.2 | 1.3 | 1.1 | 1.2 | 1.1 | 2.4 |
| Radiolucency | 0.33 | 0.33 | 0.32 | 0.30 | 0.30 | 0.21 |
| Dentine Adhesion | 32 | 29 | 31 | 30 | 30 | 22 |
| strength (MPa) | | | | | | |
| Cytotoxicity (RI) | 0/1 | 0/1 | 0/1 | 0/1 | 0/1 | 1/1 |

As shown in Table 3, the light-curing adhesive composition comprising the prepolymer mixture of Bis-GMA with Tri-GMA and/or Tetra-GMA in accordance with the present invention has better physical and mechanical properties, such as low polymerization shrinkage and water absorption due to hydroxy group blocking, convenience for use by using the reduced amount of diluent due to low viscosity, and superior adhesion strength on teeth, compared to the conventional light-curing adhesive composition comprising only the Bis-GMA prepolymer. Furthermore, the prepolymer mixture of Bis-GMA with Tri-GMA and/or Tetra-GMA can cause the adhesion strength on teeth greater than that which is found in a Tri-GMA- or Tetra-GMA-based dental adhesive composition.

Particularly, the dental adhesive composition of Example 1 based on the 50:50 wt % mixture of Bis-GMA and Tri-GMA has more improved biocompatibility as well as the best physical and mechanical properties.

Thus, the light-curing adhesive composition in accordance with the present invention has better physical and mechanical properties, such as photoconversion, polymerization shrinkage, water absorption and solubility, radiolucency and adhesion strength and shows little cytotoxicity.

While the present invention has been shown and described with particular examples, it will be apparent to those skilled in the art that many changes and modifications may be made without departing from the spirit and scope of the invention as defined in the appended claims.

What is claimed is:

1. Light-curing dental adhesive compositions, which show improved physical and mechanical properties, and adhesive performance, comprising:
(a) 1 to 50 wt % of the prepolymer mixture selected from a group consisting of a mixture of 2,2-bis-(4-(2-hydroxy-3-methacryloyloxypropoxy)phenyl)propane (Bis-GMA) of formula 1 with trifunctional methacrylate (Tri-GMA) of formula 2, a mixture of Bis-GMA With tetrafunctional methacrylate (Tetra-GMA) of formula 3 and a mixture of Bis-GMA, Tri-GMA and Tetra-GMA,
(b) 1 to 40 wt % of an adhesive monomer;
(c) 1 to 10 wt % of a hydrophilic monomer;
(d) 0.1 to 10 wt % of a photoinitiation system; and
(e) 10 to 60 wt % of a diluent,
wherein the wt % of all the components are based on the total weight of the composition;

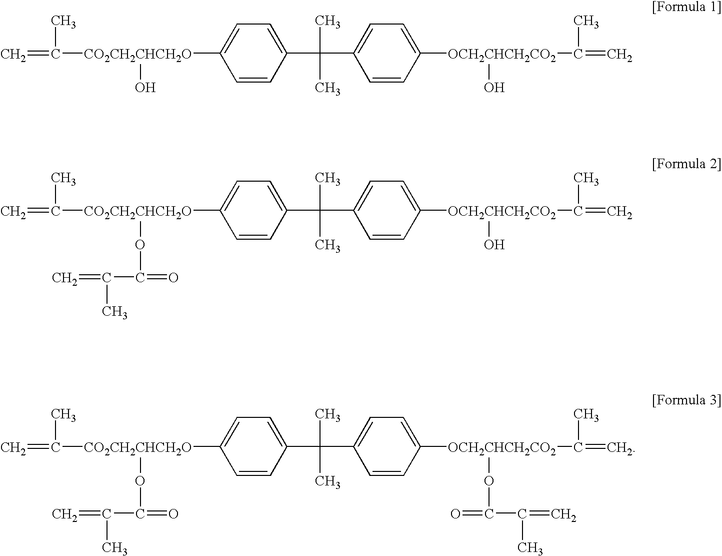

2. The Light-curing dental adhesive compositions according to claim 1, wherein the prepolymer mixture is the mixture of 95 to 5 wt % of Bis-GMA with 5 to 95 wt % of Tri-GMA based on the weight of the prepolymer mixture.

3. The Light-curing dental adhesive compositions according to claim 1, wherein the prepolymer mixture is the mixture of 95 to 5 wt % of Bis-GMA with 5 to 95 wt % of Tetra-GMA based on the weight of the prepolymer mixture.

4. The Light-curing dental adhesive compositions according to claim 1, wherein the prepolymer mixture is the mixture of 90 to 5 wt % of Bis-GMA with 90 to 5 wt % of Tri-GMA and 90 to 5 wt % of Tetra-GMA based on the weight of the prepolymer mixture.

5. The Light-curing dental adhesive compositions according to claim 1, wherein the adhesive monomer is selected from a group consisting of (meth)acrylic acid, maleic acid, p-vinylbenzoic acid, 11-(meth)acryloxy-1,1-undecandicarboxylic acid, 1,4-di(meth)acryloyloxyethylpyromellitic acid, 6-(meth)acryloyloxyethyl naphthalene-1,2,6-tricarboxylic acid, 4-(meth)acryloyloxymethyltrimellitic acid and its anhydride, 4-(meth)acryloxyethyltrimellitic acid and its anhydride, 4-(meth)acryloxybuthyltrimellitic acid and its anhydride, 4-[2-hydroxy-3-(meth)acryloyloxy]buthyltrimellitic acid and its anhydride, 2,3-bis(3,4-dicarboxybenzoyloxy)propyl(meth)acrylate, 2-, 3- or 4-(meth)acryloyloxybenzoic acid, N—O-di(meth)acryloyloxytyrosine, O-(meth)acryloyloxytyrosine, N-(meth)acryloyloxytyrosine, N-(meth)acryloyloxyphenylalanine, N-(meth)acryloyl-p-aminobenzoic acid, N-(meth)acryloyl-O-aminobenzoic acid, an addition product of glycidyl(meth)acrylate with N-phenylglycine or N-tolylglycine, 4-[(2-hydroxy-3-(meth)acryloyloxypropyl)amino]phthalic acid, 3- or 4-[N-methyl-N-(2-hydroxy-3-(meth)acryloyloxy)amino]phthalic acid, (meth)acryloylamino salicylic acid, (meth)acryloyloxy salicylic acid, (meth)acrylate of 3,3,4,4′-benzophenone tetracarboxylic dianhydride (BTDA) or 3,3,4,4-diphenyltetracarboxylic dianhydride, 2-(3,4-dicarboxylbenzoyloxy) 1,3-di(meth)acryloyloxypropane, 2-(meth)acryloyloxyethyl phosphate, 2- and 3-(meth)acryloyloxypropyl phosphate, 4-(meth)acryloyloxybuthyl phosphate, 6-(meth)acryloyloxyhexyl phosphate, 8-(meth)acryloyloxyoctyl phosphate, 10-(meth)acryloyloxydecyl phosphate, 12-(meth)acryloyloxydodecyl phosphate, bis[2-(meth)acryloyloxyethyl]phosphate, bis[2-(meth)acryloyloxyethylphenyl]phosphate, bis[2-(methyl)acryloyloxyethyl p-methoxyphenyl]phosphate, 2-sulfoethyl(meth)acrylate, 2- or 1-sulfo-1 or 2-propyl(meth)acrylate, 1- or 3-sulfo-2-butyl(meth)acrylate, 3-bromo-2-sulfo-2-propyl(meth)acrylate, 3-methoxy-1-sulfo-2-propyl(meth)acrylate, 1,1-dimethyl-2-sulfoethyl(meth)acrylamide propanesulfonic acid and 2-methyl-2-(methyl)acrylamide propane sulfonic acid and a mixture thereof.

6. The Light-curing dental adhesive compositions according to claim 1, wherein the hydrophilic monomer is selected from a group consisting of hydroxyethyl methacrylate, hydroxypropyl methacrylate and a mixture thereof.

7. The Light-curing dental adhesive compositions according to claim 1, wherein the photoinitiation system comprises 0.05 to 5 wt % of a photoinitiator and 0.05 to 5 wt % of a reductant, the photoinitiator is camphorquinone and the reductant is N,N-dimethylaminoethyl methacrylate or ethyl p-dimethyl aminobenzoate.

8. The Light-curing dental adhesive compositions according to claim 1, wherein the diluent is selected from a group consisting of ethyl alcohol, acetone and water.
